(19) United States
(12) Patent Application Publication
(10) Pub. No.: US 2022/0203109 A1
(43) Pub. Date: Jun. 30, 2022

(54) MONITORING DEVICE AND METHOD

(71) Applicant: LOIS MEDICAL LIMITED, Painswick, Stroud (GB)

(72) Inventors: Kristian James Colin WEBB, Painswick, Stroud (GB); Patrick Christopher Phillips, Painswick, Stroud (GB); Steven John MCEVOY, Painswick, Stroud (GB)

(73) Assignee: LOIS MEDICAL LIMITED, Painswick, Stroud (GB)

(21) Appl. No.: 17/606,326

(22) PCT Filed: Apr. 21, 2020
(86) PCT No.: PCT/GB2020/050987

§ 371 Date: Oct. 25, 2021

(30) Foreign Application Priority Data
May 3, 2019 (GB) 1906290.0

(51) Int. Cl. A61N 1/39; A61B 5/00; A61N 1/37; A61N 1/372; G08B 21/02

(52) U.S. Cl. CPC A61N 1/3937; A61B 5/0028; G08B 21/02; A61N 1/37282; A61N 1/3706

(57) ABSTRACT

A monitoring device comprising: electric field detection circuitry configured to detect an electric field; and processing circuitry configured to: determine whether the detected electric field has a characteristic indicative of a predetermined electric field source; and if the detected electric field has the characteristic, output a signal indicating that the predetermined electric field source has been detected.

### BACKGROUND

### Field of the Disclosure

The present invention relates to a monitoring device and method.

### Description of the Related Art

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present invention.

As medical devices have improved in recent years, it is now possible for such devices to be implanted inside the body of a user so as to detect certain abnormalities in the physiological function of that user's body and, in some cases, to perform corrective action in response to detecting such an abnormality. An example of such a medical device is an implantable cardioverter defibrillator (ICD). ICDs are defibrillators designed to treat a cardiac arrest in patients that have, for example, either survived a previous cardiac arrest or patients who have a heart condition that increases the risk of a cardiac arrest occurring. ICDs have the ability to treat a cardiac arrest by converting a dangerous arrhythmia of the heart with a high energy electrical impulse (otherwise known as an electric shock). During the shock, the patient will either experience a large “kick” to the chest (if conscious) or will be unaware of the therapy (due to a loss of consciousness).

Implantable medical devices such as ICDs provide a very fast response to physiological abnormalities in a user which, if left untreated, may result in the patient becoming very unwell (perhaps even fatally). A problem, however, is that although, although medical devices such as ICDs are able to instantly attempt to treat a physiological problem such as a cardiac arrest (following a therapy provided by the device) it is usually still necessary for the user concerned to take action to inform others of the event and/or to alert a doctor or other healthcare professional in order to allow the cause of the abnormal physiological behavior to be investigated and to determine whether ongoing treatment needs to be introduced or reviewed in order to reduce the risk of the physiological abnormality happening again. In addition, it can be the case that, despite treatment administered automatically by the implanted medical device, the physiological abnormality may not be sufficiently treated by the implanted medical device. For example, an ICD, despite providing several shocks to a user, may not be able to effectively treat a dangerous arrhythmia. In this case, the patient will often be unable to seek help (due to loss of consciousness, for example). Such a situation is particularly dangerous if the patient is alone (since if external help by a third party is not received, the patient is likely to experience a fatal cardiac arrest).

Medical devices such as ICDs have been introduced which are configured to communicate with a base station positioned in the home of the wearer. Such systems are designed to flag any abnormalities and to reduce the number of outpatient appointments the patient has to attend. The base station receives wireless signals (e.g. radio signals) from the medical device implanted in the patient's body and, in turn, forwards the signals (e.g. over the internet) to a healthcare provider. The data extracted from the implanted device and transmitted by the home base station is determined according to a preset specification. This nominally includes device settings, device function, measurements, physiological abnormalities and any therapies that have been administered. For example, in the case of an ICD, the ICD may transmit signals to a home base station indicating the current cardiac rhythm of the wearer and data on any previous rhythm abnormalities and any electric shocks provided by the ICD to treat dangerous arrhythmias.

A problem with such existing systems, however, is that the data fed to the system of a hospital or healthcare professional by the base station is merely a data recording tool which becomes associated with a patient's medical record or the like. That is, although data is received from a medical device such as an ICD implanted in the body of the patient, this data is used to monitor a patient's condition generally and to help guide the specifics of ongoing treatment of the patient. Thus, if, for example, the patient is suffering a cardiac arrest which cannot be successfully treated by an implanted medical device such as an ICD, a doctor or other healthcare professional will not know this unless the medical records of the patient are accessed manually. Typically, manual access of a patient's medical records including received medical device data is not carried out as a matter of routine but, rather, is accessed when providing treatment or check-up appointments to the patient. Thus, the fact that a patient has suffered a potentially fatal physiological abnormality such as a cardiac arrest will not be known by anyone until the patient's medical records are checked. If the patient has not been able to seek help themselves (e.g. if they are alone in their home and are physically unable to call for help), then it may be too late to save the patient's life. Another problem associated with current systems is that they will only work when the user is in their home. This is because of the need for signals transmitted by implanted medical devices such as ICDs to be transmitted to a hospital or healthcare provider via a base station installed in the home.

There is a need to alleviate the above-mentioned problems.

### SUMMARY

The present technique is defined by the claims.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 schematically shows a system according to an embodiment of the present technique;

FIGS. 2A and 2B each schematically illustrate an example of information displayed by an alert device;

FIG. 3 schematically shows a first example of the present technique according to an embodiment;

FIG. 4 schematically shows a second example of the present technique according to an embodiment;

FIGS. 5A and 5B schematically show, respectively, a transmitter of an implanted medical device and a receiver of a wearable device, according to an embodiment;

FIG. 6 schematically shows a sequence of events when the present technique is used with an implanted cardioverter defibrillator, according to an embodiment;

FIG. 7 schematically shows a method of operating a wearable device according to an embodiment;

FIG. 8 schematically shows a method of operating an implanted medical device according to an embodiment;

FIG. 9 schematically shows a method of operating an alert device according to an embodiment;

FIG. 10 schematically shows a patient monitoring device according to a further embodiment;

FIG. 11 schematically shows an electric field detector of the patient monitoring device according to the further embodiment;

FIG. 12 schematically shows circuitry components of the electric field detector according to the further embodiment;

FIG. 13 schematically shows the effect of an ICD electric field on the electric field detector according to the further embodiment;

FIGS. 14A to 14C schematically show a voltage signal output by a charge amplifier of the electric field detector in response to the ICD electric field according to the further embodiment;

FIG. 15 schematically shows the voltage signal output by the charge amplifier in more detail;

FIG. 16 schematically shows a user interface of the patient monitoring device according to the further embodiment; and

FIG. 17 shows a method according to the further embodiment.

### DESCRIPTION OF THE EMBODIMENTS

Referring now to the drawings, wherein like reference numerals designate identical or corresponding parts throughout the several views.

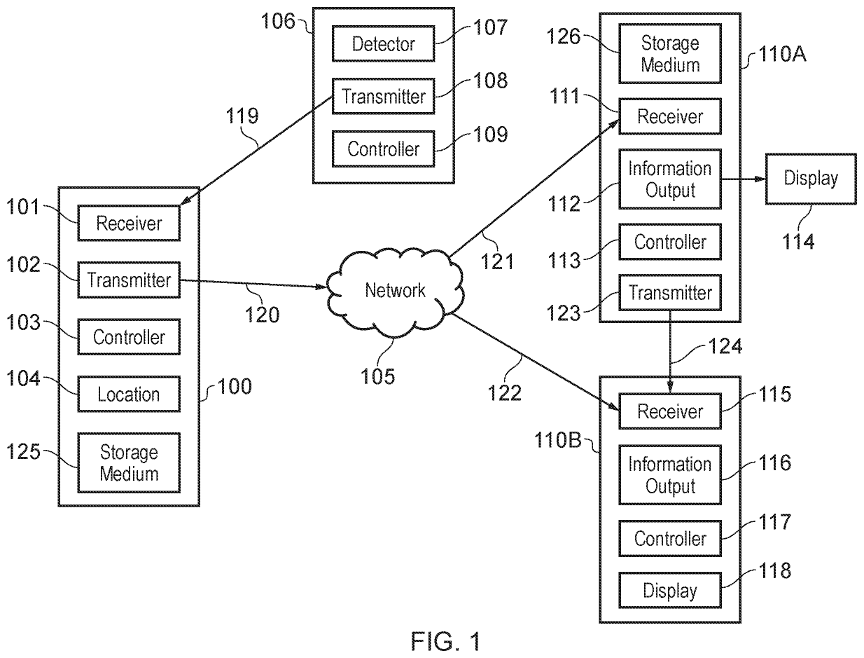

FIG. 1 shows a system according to an embodiment of the present technique. The system comprises a medical device 106 which is implantable in the body of a user, a wearable device 100 which is wearable by the user (for example, as a bracelet on a wearer's wrist or ankle, a ring on the user's finger, an item of clothing warn by the user or an adhesive patch adhered to the user's skin), and a plurality of alert devices 110A and 110B. In other embodiments, it will be noted that there may be only one alert device.

The medical device 106 is a device such as an ICD which is implantable in a body of a user (the user is not shown in FIG. 1). The medical device 106 comprises a detector 107 configured to detect, when the medical device is implanted in the user's body, a predetermined physiological event (such as an abnormal arrhythmia indicative of a potential cardiac arrest). The medical device may operate any known technology for detecting the predetermined physiological event concerned. The technology associated with detecting predetermined physiological events such as cardiac arrests is well known and therefore will not be described in detail here. An example of an ICD that implements such technology and which may be used with embodiments of the present technique is the Medtronic® Visia ICD.

The medical device 106 also comprises a transmitter 108 configured to transmit, in response to the detector 107 detecting a predetermined physiological event, a signal to the wearable device 100 worn by the user. The transmission of the signal from the transmitter 108 of the medical device 106 to a receiver 101 of the wearable device 100 is shown by arrow 119. The operation of the detector 107 and transmitter 108 is controlled by the controller 109. Each of the detector 107, transmitter 108 and controller 109 may be implemented using circuitry, for example. In particular, the controller 109 may comprise processing circuitry for processing instructions to control the detector 107 and transmitter 108.

The wearable device 100 comprises a receiver 101 configured to receive the signal transmitted by the transmitter 108 of the medical device 106. The wearable device 100 also comprises a transmitter 102 configured to transmit, in response to the receiver 101 receiving the signal from the implanted medical device 106, alert information to one or more of the alert devices 110A and 110A for alerting a person other than the wearer of the wearable device of the occurrence of the predetermined physiological event detected by the detector 107 of the medical device 106. The wearable device 100 also comprises location circuitry 104 for determining a position of the wearable device (and therefore of a user wearing the wearable device). The wearable device also comprises a storage medium 125 (comprising semi-conductor memory, for example) for storing digital information. Each of the receiver 101, transmitter 102, location circuitry 104 and storage medium 125 are controlled by a controller 103. Each of the receiver 101, transmitter 102, controller 103 and location circuitry 104 may be implemented as circuitry, for example. In particular, the controller 103 may comprise processing circuitry configured to process instructions for controlling the receiver 101, transmitter 102, location circuitry 104 and storage medium 125.

The alert device 110A comprises a receiver 111 configured to receive, from the wearable device 100, alert information, the alert information being transmitted by the transmitter 102 of the wearable device 100 in response to the wearable device 100 receiving the signal transmitted along arrow 119 by the transmitter 108 of the medical device 106. In this example, the alert information transmitted to the alert device 110A is transmitted via a communications network 105 according to the arrows 120 and 121. In embodiments, the communications network 105 may be the internet, for example. The alert device 110A also comprises information output circuitry 112 configured, in response to the receiver 111 receiving the alert information, to output information indicative of the occurrence of the predetermined physiological event to a person other than the user wearing the wearable device 100. In this example, the output information is output to a display 114 (such as a liquid crystal display (LCD) or the like) which is separate to the alert device 110A (e.g. the display 114 may be a display of a separate computer monitor). The wearable device also comprises a storage medium 126 (comprising semi-conductor memory, for example) for storing digital information The receiver 111, information output circuitry 112 and storage medium 126 are controlled by a controller 113. Each of the receiver 111, information output circuitry 112 and controller 113 may be implanted using circuitry, for example. In particular, the controller 113 may comprise processing circuitry configured to process instructions for controlling the receiver 111, information output circuitry 112 and storage medium 126. In one example, the alert device 110A is a computer located at the premises of a healthcare provider (such as a clinic, hospital, monitoring service or insurance provider). The received alert information may therefore be used by the information output circuitry 112 to generate output information for display on the display 114 for alerting a healthcare professional (such as a doctor or nurse) of information associated with the predetermined physiological event detected by the medical device 106.

The alert device 110B has similar functionality to the alert device 110A. In particular, the alert device 110B comprises a receiver 115, information output circuitry 116 and a controller 117 which operate in the same way as the receiver 111, information output circuitry 112 and controller 113 of the alert device 110A. However, this time, a display 118 (such as an LCD display or the like) is comprised as part of the alert device 110B. The alert device 110B may therefore be a terminal device such as a mobile phone, tablet computer, laptop computer or the like which comprises a built-in display 118. In the case of the alert device 110B, the receiver 115 receives the alert information from the wearable device 100 over the network, as indicated by arrows 120 and 122. The alert device 110B may be a terminal device owned by a person (such as a friend or relative) previously chosen by the wearer of the wearable device 100 (that is, chosen in advance) so as to enable them to be alerted of any predetermined physiological event detected by the medical device 106.

In the example of alert device 110B, alert information received at the receiver 115 is used by the information output circuitry 116 to generate output information for display on the display 118 to alert the chosen person in the event of a predetermined physiological event occurring. For example, the terminal device may be owned by a friend or relative of the wearer who has been chosen by the wearer to be alerted in response to the occurrence of the predetermined physiological event. It will be appreciated that there may be more alert devices than the alert devices 110A and 110B shown in FIG. 1. It will also be appreciated that the route through which alert information is transmitted through the network 105 may be different to that shown.

For example, the alert information may initially be transmitted from the transmitter 102 of the wearable device 100 to the first alert device 110A via the arrows 120 and 121. The alert information (which may be edited by the alert device 110A) may then be transmitted to the alert device 110B (for receipt at the receiver 115 of the alert device 110B) instead of the alert information being directly transmitted to the alert device 110B from the wearable device 100. Such a scenario may be appropriate when, for example, the alert device 110A is comprised as part of a central server located at the premises of a healthcare provider such as a clinic or hospital and the alert device 110B is a terminal device of a friend or relative of the wearer of the wearable device 100. In this case, the alert information indicating that the predetermined physiological event detected by the medical device 106 has occurred is first received by the healthcare provider so that they may begin taking appropriate action, such as asking the wearer of the wearable device 100 (who is also the user within whose body the medical device 106 has been implanted) to attend an appointment for a check-up or, in more serious cases, to call an ambulance or the like so as to provide emergency treatment to the wearer of the wearable device 100. The alert information is then forwarded to the alert device 110B (e.g. via the network 105) in order to alert the friend or relative that the predetermined physiological event has occurred and that the wearer of the wearable device 100 (who may be referred to as the patient) is now being provided with assistance.

It will be appreciated that other configurations for the transfer of information between a plurality of alert devices such as 110A and 110B may be implemented. In any case, it will be appreciated that a receiver of an alert device (such as receivers 111 and 115 of alert devices 110A and 110B, respectively) is able to receive alert information either directly from the wearable device 100 over the network 105 or, alternatively, may receive the alert information from another alert device which has previously received the alert information directly from the wearable device 100 over the network 105. The alert information forwarded from a first alert device 110A to a second alert device 110B is based on the alert information received from the wearable device 100. For example, in the case that alert information is received from the wearable device 100 by an alert device 110A comprised within a hospital or clinic, the alert information may comprise information indicating that a cardiac arrest has occurred, together with other information such as how long the cardiac arrest has been occurring for, the number of shocks that have been output by the medical device 106 (in this case, an ICD), the intensity of each shock (e.g. the electrical energy delivered to the patient during each shock, in joules) and any other relevant information associated with the cardiac arrest and the treatment which has been applied by the medical device 106. This detailed information is appropriate for a doctor or healthcare professional, but not necessarily appropriate for a friend or relative of the patient (who may not be medically trained and who therefore may not understand the detailed information provided as part of the alert information). The alert information forwarded to the alert device 110B may therefore be edited by the controller 113 of the alert device 110A prior to the alert information being forwarded in order to make the information displayed on the display 118 of the alert device 110B more appropriate. For example, in the case that the alert information is to be forwarded to the alert device of a relative or friend of the patient (who may not be medically trained and may therefore not understand detailed medical information), the alert information may be simplified prior to being forwarded so as to only include key details which are more understandable to a non-professional. For example, the output information may simply indicate that a cardiac arrest has occurred and that treatment administered by the medical device 106 has failed, without providing specific technical details of the cardiac arrest (as detected by the medical device 106) or the treatment provided (such as the frequency and energy of shocks provided by the medical device 106). The forwarded alert information may also include additional information such as the action taken by the healthcare provider (e.g. information indicating that an ambulance is on its way to the patient). The alert information transmitted by the wearable device 100 may therefore be edited (e.g. amended, reduced or expanded) before being forwarded from one alert device 110A to another alert device 110B in accordance with the intended audience of the alert information. The alert information (whether or not it has been edited) is transmitted from a transmitter 123 comprised within the alert device 110A to the receiver 115 of the alert device 110B in the direction shown by arrow 124. Although not shown (for the sake of clarity of FIG. 1), the alert information may be transmitted from the alert device 110A to the alert device 110B via the network 105. The transmitter 123 may be implemented as circuitry and is controlled by the controller 123. The receiver 111, transmitter 123, controller 113 and storage medium 126 of the alert device 110A allow the alert device 110A to act as a server in the system shown in FIG. 1, the alert device 110A managing the distribution of alert information transmitted to it from the wearable device 100 over the network 105 to other alert devices (such as alert device 110B). The alert device 110A may perform other functions of a server, such as storing information in the storage medium 126 (such as information associated with the patient 300 wearing the wearable device) for use in editing alert information before it is forwarded to another alert device and routing alert information to the correct one or more wearable devices (for example, information indicative of one or more alert devices such as alert device 110B belonging to previously chosen friends or relatives of the patient may be stored in the storage medium 126).

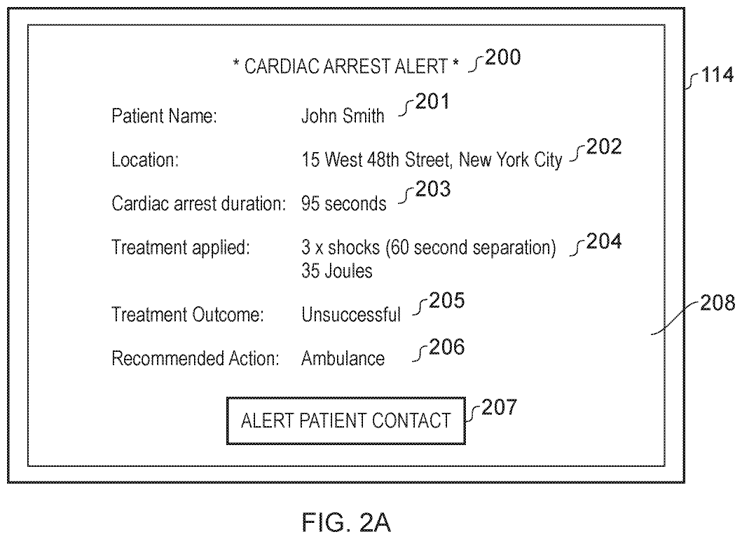

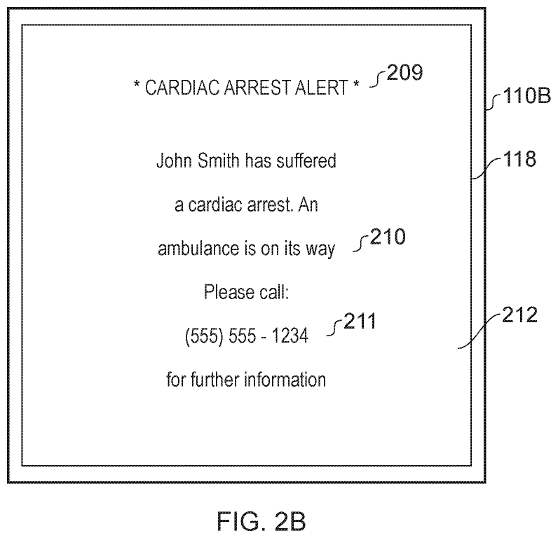

FIGS. 2A and 2B illustrate an example of the information displayed by an alert device 110A (e.g. located at a hospital) and the information output by an alert device 110B (e.g. a mobile phone of a friend or family member of the patient) in response to the occurrence of a predetermined physiological event detected by the medical device 106. In the examples of FIGS. 2A and 2B, the medical device is an ICD which detects and attempts to treat a cardiac arrest.

FIG. 2A shows a display 114 displaying information output by the information output circuitry 112 in response to alert information being received by the receiver 111. The output information comprises information indicating the predetermined physiological event which has been detected. This is the information 200. In this case, the detected physiological event is a cardiac arrest. Also output is the patient's name 201, the current location of the patient 202 (as determined by the location circuitry 104 of the wearable device 100, for example), a duration of the cardiac arrest 203 (as measured by the detector 107), the treatment applied 204 (in this case, that three electric shocks have been provided to the user, each of these having an energy of 35 joules and each shock being 60 seconds apart), an outcome of the treatment 205 (which in this case, is “unsuccessful”—that is, the cardiac arrest has not been alleviated by the shocks) and a recommend action 206 (which, in this case, is for an ambulance to be called to the patient's location). Each of these pieces of information is comprised within the received alert information and is displayed as part of an image 208 displayed on the display 114. In addition, a virtual button 207 is also displayed as part of the image 208. The virtual button is selectable by a viewer of the display 114 (in particular, a doctor or other healthcare professional working at the hospital at which the alert device 110A is located) in order to initiate the forwarding of the alert information to the alert device 110B held by the friend or relative of the patient. The viewer of the display 114A selects (that is, operates) the virtual button 207 via any suitable user interface (not shown) of the alert device 110A, such as a mouse, keyboard, touchscreen or the like.

In response to the selection of the virtual button 207, the alert information is forwarded to the alert device 110B (e.g. via the network 105). In response to the receiver 115 receiving the alert information, information indicating the occurrence of the cardiac arrest 209, more detailed information about the patient 210 (in particular, the patient name and the action taken so far, namely that an ambulance is on its way) and a telephone number 211 are provided as part of an image 212 displayed on the display 118 of the alert device 110B. This information alerts the friend or relative previously chosen by the patient to be alerted in the case of a cardiac arrest to be notified that the cardiac arrest has occurred, some essential information regarding who has suffered the cardiac arrest, what action has been taken and what to do next (in this case, to call the given telephone number 211). In this case, the alert device 110B is a smartphone of the friend or relative of the patient.

In the above example, by the doctor (or other healthcare professional) selecting the virtual button 207, it is assumed that the recommend action of calling an ambulance has been carried out. This is why the information 210 displayed on the display 118 of the alert device 110B indicates that an ambulance is on its way. In other embodiments, it would be appreciated that the operator of the alert device 110A may edit the message to be displayed to the user of the alert device 110B prior to the alert information being forwarded to the alert device 110B. This allows a doctor or other health care professional to provide any further information which might be useful or relevant to the viewer of the alert device 110B (in this way, the doctor is able to edit the alert information before it is forwarded to the alert device 110B).

It will thus be appreciated that, generally, the alert information transmitted by the transmitter 102 of the wearable device 100 may be used by one or more alert devices (such as alert devices 110A and 110B) in any number of ways. Generally, the alert information is information for alerting a person other than the patient of the occurrence of a predetermined physiological event such as a cardiac arrest as detected by an ICD. This alert information may then be used in different ways by different respective alert devices. Furthermore, the alert information may be transmitted to one alert device via another alert device so as to enable the distribution of the alert information to be controlled and to enable the alert information to be amended (e.g. by the removal or addition of certain details) depending on the type of user of the alert device to which the alert information is to be forwarded. Alternatively, each alert device may be provided with the same alert information via the network 105 and a controller of each respective alert device may then control the information output to the user of that device based on the received alert information and on the type of user (e.g. professional or non-professional) of that alert device.

It will thus be appreciated that each alert device may receive alert information directly from the wearable device 100 over the network 105 or, alternatively, may receive alert information from another alert device. The alert information may be amended by the transmitting alert device prior to the alert information being transmitted to the receiving alert device so as to enable information appropriate to the user of each respective alert device to be displayed on that device. As long as the amended alert information still comprises information for alerting a user of each respective alert device of the occurrence of the predetermined physiological event (such as a cardiac arrest in the example of FIGS. 2A and 2B), then the purpose of the alert information is conserved. That is, information transmitted to one alert device from another alert device remains alert information as long as the information is for alerting each user of the respective one or more receiving alert devices of the occurrence of the predetermined physiological event detected by the medical device 106 implanted in the body of the patient.

It will be appreciated that information output by the output circuitry 112 and 116 of the alert devices 110A and 110B, respectively, may be output to another type of device for providing cognitively comprehensible information to a user. For example, instead of or in addition to the output information being displayed on a display (such as an LCD display or the like), the information may be output by a sound processor and loudspeaker (not shown) in order to alert the user of a particular alert device of the occurrence of the predetermined physiological event detected by the medical device 106.

In the example of FIG. 1, the receiver 115 of the alert device 110B may be configured to receiver alert information both directly from the wearable device 100 (via the path indicated by arrows 120 and 122) and via the alert device 110A (as indicated by the arrows 120, 121 and 124). Such an arrangement allows the user of the alert device 110B to be immediately notified of the occurrence of the predetermined physiological event detected by the medical device 106. Further information (e.g. that an ambulance has been called) may then be received at a later point from the alert device 110A, thus providing the user of the alert device 110B with more comprehensive information about the predetermined physiological event and the actions that have been taken. This allows the user of the alert device 110B to be provided with information regarding the occurrence of the predetermined physiological event as soon as that information becomes available. This allows, for example, the user of the alert device 110B to help the patient (if they are near by) prior to being informed by the user of the alert device 110A (e.g. the hospital) that an ambulance is on its way. In this case, different portions of alert information are received at different times from different sources, depending on when and from where that alert information becomes available.

It will be appreciated that each alert device may be implemented using conventional hardware using an appropriate software application or the like. For example, the alert device 110A may be a personal computer located within a hospital or clinic running a software application which controls the personal computer to carry out the functions of the receiver 111, information output circuitry 112, controller 113 and transmitter 123 (where present). Similarly, the alert device 110B may be a terminal device such as a mobile phone, tablet computer or the like running a software application which controls the terminal device to carry out the functions of the receiver 115, information output circuitry 116, controller 117 and display 118. In this case, the software application may be downloaded as an “app” (e.g. as compatible with Android® devices or iOS® devices). In such cases, the function of each alert device as described is implemented using the existing communication and processing hardware of the device on which the software application is installed.

In the example of FIGS. 2A and 2B, in the case that a first instance of alert information is received by the alert device 110B directly from the wearable device 100 and that a second instance of alert information is received, at a subsequent time, from the alert device 110A, it may be the case, for example, that, initially, in response to the receipt of the first instance of alert information, information 209 indicating that a cardiac arrest has occurred, together with the telephone number 211 is displayed on the display 118. Then, at a later time, when the second instance of alert information is received (e.g. after a medical professional operating the alert device 110A has reviewed the information shown in FIG. 2A and selected the button 207), further information indicating that an ambulance has been called is displayed on the display 118. It will thus be appreciated that information displayed on the display 118 of the alert device 110B (such as a smartphone of a friend or relative of the patient) may be updated using a plurality of instances of alert information received from different sources (e.g. directly from the wearable device 100 and from another alert device 110A) at different times, depending on when this alert information becomes available.

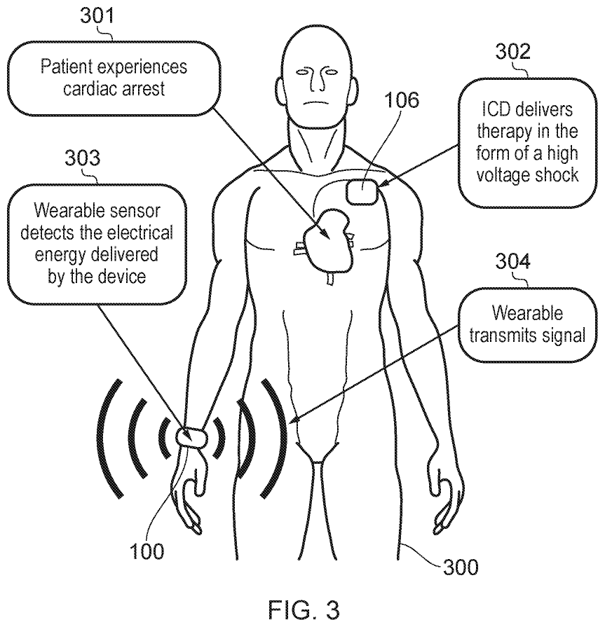

FIG. 3 shows an example of the present technique according to one embodiment. In this case, the medical device 106 is an ICD and the signal transmitted by the medical device 106 in response to detection of a potential cardia arrest in the patient is the electric signal generated as a result of an electric shock given to the patient 300. The transmitter 108 of the medical device 106 is therefore an electrode which delivers the electric shock to the patient 300 in order to attempt to treat the cardiac arrhythmia. FIG. 3 shows various events which occur during and following detection of such a potential cardiac arrest. At step 301, the patient experiences a dangerous cardiac arrhythmia indicative of a cardiac arrest. At step 302, in response to the detector 107 of the ICD detecting the dangerous cardiac arrhythmia (according to known techniques in existing ICDs), the ICD delivers a high voltage shock to the chest of the patient 300. The electrical energy resulting from the application of the electric shock travels through the organic tissue (body tissue) of the patient 300 and is detected by the wearable device 100 at step 303 (in particular, the electrical energy is detected by the receiver 101 of the wearable device 100). The signal detected by the receiver 101 is therefore the electrical energy of the applied electric shock which has travelled to the wearable device 100 (in this case, a bracelet worn around the wrist of the patient 300) through the organic tissue of the patient. The receiver 101 therefore comprises a component for detecting the presence of electric energy, for example, a voltmeter which detects the voltage (potential difference) between the patient's skin and a portion of the wearable device 100 not in contact with the patients skin. In the case that the detected voltage increases by more than a predetermined amount (optionally, within a predetermined time period), the controller 103 determines that a shock has occurred. At step 304, alert information is transmitted to one or more alert devices (such as alert devices 110A and/or 110B). The alert information is transmitted as a wireless signal, in particular, a radio signal. The alert information is then used to alert one or more appropriate persons in the way as previously described.

The alert information in embodiments may be transmitted via any suitable wireless signal to one or more predetermined alert devices.

In one example, the alert information is transmitted to one or more predetermined alert devices via a mobile telecommunications network such as a Long Term Evolution (LTE) network administered by the Third Generation Partnership Project (3GPP). In this case, the wearable device is a terminal device (or user equipment (UE)) which connects to the mobile telecommunications network in accordance with a suitable one or more of the various LTE standards. The wearable device 100 may comprise a Subscriber Identity Module (SIM) card or the like in order to enable it to connect to the mobile telecommunications network. In this case, the network 105 comprises the mobile telecommunications network so as to enable the alert information transmitted by the wearable device 100 to be received by an appropriate alert device (such as one or both of alert devices 110A and 110B).

In another example, the wearable device 100 may transmit the alert information via a connection a Local Area Network LAN (e.g. a Wireless Local Area Network WLAN such as a Wi-Fi® network). Again, in this case, the network 105 will comprise the LAN so as to enable the alert information to be transmitted to an appropriate one or both of the alert devices 110A and 110B.

It will be appreciated that the transmission of the alert information from the wearable device 100 via a mobile telecommunications network is advantageous in that the alert information may be transmitted from any location within a wide geographical area covered by mobile telecommunications networks. Furthermore, the transmission of the alert information via a local area network (e.g. a Wi-Fi® network) is advantageous in that it will still be possible to transmit alert information from locations which have no or little coverage of mobile telecommunications networks (such as certain indoor locations). The wearable device 100 (in particular the transmitter 102 of the wearable device) may be configured to transmit the alert information via either a mobile telecommunications network or local area network, depending on which of those networks are available. This allows alert information to be transmitted using the most appropriate network (e.g. the network with the best signal strength or quality) at any given time. This improves the likelihood of the alert information being successfully received by an appropriate alert device in the event of a physiological event such as a cardiac arrest.

By the wearable device 100 detecting the electric signal transmitted through the organic tissue of the user 300 as a result of an electric sock delivered by an ICD, the wearable device 100 may be used with any existing ICD implanted in the patient 300. That is, the patient does not need to have a specific ICD installed in order to benefit from the wearable device 100. Rather, any ICD (which delivers a high voltage shock in the case of a cardiac arrest) will generate (by the action of delivering a high voltage shock) an electric signal which is detectable by the wearable device 100 so as to allow alert information to be transmitted in response to the patient experiencing a cardiac arrest. The wearable device 100 may therefore work with any ICD without the need for a specific type of ICD to be implanted in the patient 300 and without any extensive configuration on the part of the user. The wearable device 100 is therefore quick and easy to set up.

In addition to an implanted medical device 106 such as an ICD omitting an electrical signal detectable by the wearable device 100 in the case of delivering a shock to the patient 300, it is also envisaged that, instead or in addition to the transmission of such an electrical signal, an implanted medical device 106 may communicate with the wearable device 100 via a wireless signal. Any wireless signal which, when transmitted by the transmitter 108 of an implanted medical device 106, is able to penetrate the organic tissue of the patient 300 so as to be received by the receiver 101 of the wearable device 100 located outside the patient's body 300 may be used. For example, a radio signal may be used.

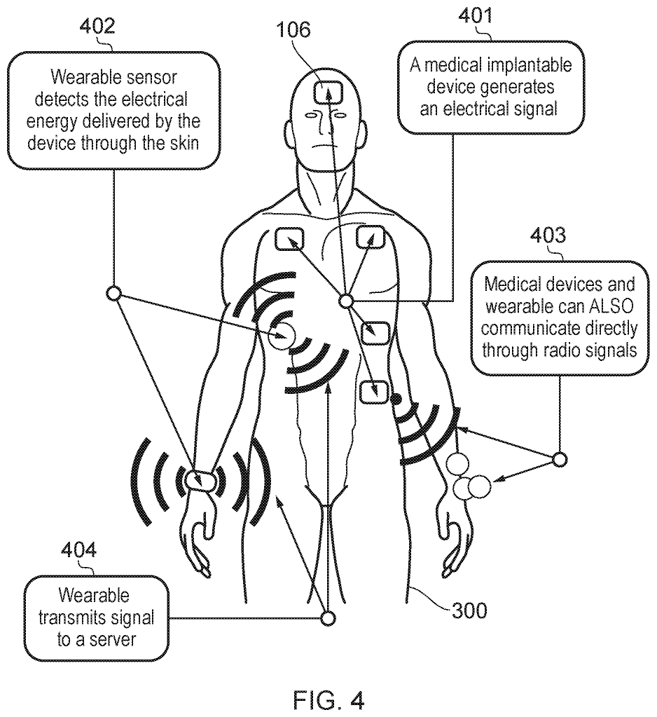

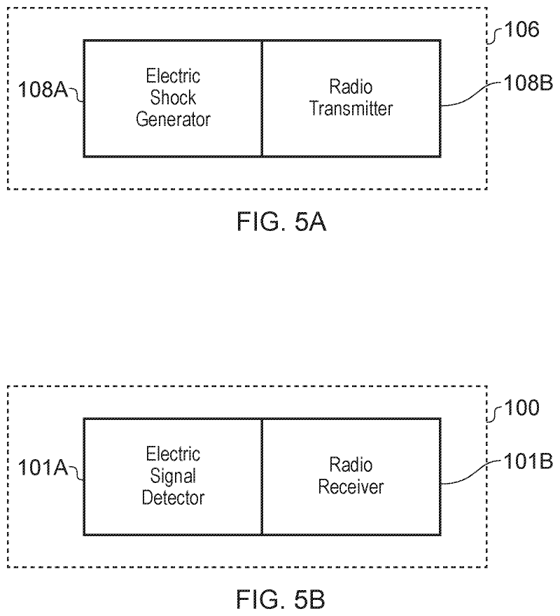

Such an arrangement is schematically illustrated in FIG. 4, in which a plurality of implantable devices 106 are implanted in the patient 300. In this case, each of the implanted devices 106 is able to transmit (a) an electrical signal to the wearable device 100 via organic tissue of the patient 300, (b) a wireless (e.g. radio) signal to the wearable device 100 or (c) transmit both an electrical signal (via the organic tissue of the patient 300) and a wireless (e.g. radio) signal to the wearable device 100. In this case, the transmitter 108 of each implanted medical display 106 is either a transmitter of an electrical signal, a transmitter of a wireless (e.g. radio) signal or comprises an electrical signal transmission portion and a wireless (e.g. radio) signal transmission portion. An example of this is schematically shown in FIG. 5A, in which the transmitter 108 of the medical device 106 comprises a first portion 108A for generating an electric shock (and therefore an electrical signal detectable by the wearable device—although not shown, the first portion 108A will comprise electrodes in contact with organic tissue of the patient's body) and a second portion 108B for transmitting radio signals. Note that, for the sake of brevity, the other components of medical device 106 are not shown in FIG. 5A.

Similarly, the receiver 101 of the wearable device 100 (a) is configured to detect an electrical signal transmitted via the organic tissue of the patient 300 (e.g. by comprising a voltmeter, as previously described), (b) is configured to detect a wireless (e.g. radio) signal transmitted by one or more of the medical devices 106 (e.g. by comprising a radio receiver) or (c) comprises both a wireless (e.g. radio) signal receiving portion and an electrical signal receiving portion. An example of (c) is schematically shown in FIG. 5B, in which the receiver 101 of the wearable device 100 comprises a first portion 101A for detecting an electrical signal (for detecting the electric energy of an electric shock provided by the medical device 106, e.g. the first portion 101A may comprise a voltmeter or the like) and a second portion 101B for receiving radio signals. Note that, for the sake of brevity, the other components of wearable device 100 are not shown in FIG. 5B.

By allowing an implanted medical device 106 to communication with the wearable device 100 via wireless signals such as radio signals (for example, a medical device 106 and wearable device 100 may be configured to exchange radio signals via a Bluetooth® or Wi-Fi® pairing between the medical device 106 and wearable device 100), the wearable device 100 is able to receive more information about the detected predetermined physiological event and any treatment applied compared to the situation of the wearable device 100 receiving an electrical signal (as a result of a shock applied to the patient by an ICD) alone. For example, a wireless signal transmitted from an ICD to the wearable device 100 may comprise additional information relating to the dangerous cardiac arrhythmia which was detected (such as data indicative of an electric cardiogram) which is received by the receiver 100 and transmitted to an appropriate alert device (such as one or both of alert devices 110A and 110B) to provide more information to healthcare professionals about the physiological event which has occurred. It will also be appreciated that, by allowing a wearable device 100 to receive wireless signals transmitted by an implanted medical device 106 in response to a detection by the implanted medical device of a predetermined physiological event, the present technique is not limited to use with medical devices such as ICD's which, by their very nature, transmit electrical signals of sufficient power such that they are detectable at another part of a patients body (e.g. the wrist, when the wearable device 100 is a wristband). For example, other types of implantable medical device 106 (such as pacemakers, implanted insulin pumps and/or glucose monitors, neuromodulation devices or any other implantable medical device for monitoring and/or treating a particular physiological condition of a patient) may transmit information via a wireless signal to the wearable device 100 so as to enable the physiological condition or event with which that medical device 106 is associated to be monitored by one or more of the patient 300 in which the device is implanted, a healthcare professional and another person nominated in advance by the patient. This allows the wearable device 100 of the present technique to be used with any implantable medical device 106 which is able to transmit information to the wearable device 100 using a wireless signal such as a radio signal.

It is noted that, in embodiments, any wireless pairing (e.g. via Bluetooth® or Wi-Fi®) of an implanted medical device 106 and wearable device 100 may be carried out according to any suitable method known in the art. Similarly, the selection of one or more alert devices to which alert information is to be transmitted over the network 105 may be carried out according to any suitable method known in the art. For example, each wearable device 100 and alert device (e.g. alert devices 110A and 110B) may be associated with unique identifier (such as a media access control (MAC) address) which enables that device to be identified on the network 105. A patient 300 (or authorised third party) may then determine the alert devices which are to be provided with alert information by configuring the wearable device 100 appropriately (e.g. using a user interface (not shown) of the wearable device 100 or the like). Configuring a particular device to communicate certain information to one or more other particular devices via a communications network is known in the art, and will therefore not be discussed in detail here.

As previously mentioned, the alert information may comprise data associated with the predetermined physiological event detected by the implanted medical device 106. This data is based on the signal received from the implanted medical device, whether this signal is an electrical signal (e.g. generated by an ICD delivering an electric shock to a patient 300 who is experiencing a potential cardiac arrest) or a wireless signal such as a radio signal. Examples of the data included in the alert information are discussed with reference to FIGS. 2A and 2B, for example (e.g. in the case of FIG. 2A, the alert information comprised information indicative of the patient name 201, the cardiac arrest duration 203, the treatment applied 204, the treatment outcome 205 and a recommended action to be taken 206). A portion of information comprised within the alert information may be determined by the controller 103 based on the signal received by the receiver 101 from the medical device 106. For example, if the signal is an electric signal resulting from an electric shock applied to the user, then information indicative of the cardiac arrest duration 203 and treatment applied 204 may be determined by the controller 103 based on the received electric signal (e.g. the cardiac arrest duration 203 may be determined from the time duration over which shocks have been applied to the patient 300 and the treatment applied 204 (e.g. number of shocks, time between shocks and the energy of each shock) may be determined based on the number of electric signals detected and their energy). It will be appreciated that even more information to be included in the alert information may be received from the medical device 106 in the case that a wireless (e.g. radio) signal is used to transmit this information from the medical device 106 to the wearable device 100 (wireless signals such as radio signals can carry larger amounts of data than a single electrical pulse resulting from the application of an electric shock to the patient). Alternatively, or in addition, a portion of information comprised within the alert information may be stored in the storage medium 125 of the wearable device. For example, information indicative of the patient name 201 may be stored in the storage medium and included in the transmitted alert information. This information may be the patient name itself, or, alternatively, may simply be a unique identifier of the wearable device (e.g. a MAC address) which enables the patient's name to be looked up by a server (e.g. alert device 110A, which also functions as a server in some embodiments) of the network 105 in which the patient's name is stored in advance. The looking up of data at a server based on a unique identifier of a particular hardware device is known in the art, and will therefore not be described in detail here. It is also noted that the storage medium 125 of the wearable device 100 may also store information indicative of the alert devices to which the alert information is to be sent (e.g. the MAC addresses of those devices). Alternatively, in the case that alert information is first transmitted to a server (such as alert device 110A) prior to being forwarded to one or more other alert devices, information indicative of the one or more other alert devices may be stored in storage medium of the server (such as storage medium 126 of alert device 110A).

In general, it will be appreciated that alert information (which, in one embodiment, may simply comprise a signal indicating that the predetermined physiological event has occurred or, in an alternative embodiment, may comprise one or more further types of information associated with the predetermined physiological event, such information being comprised within the signal received by the receiver 101 from the implanted medical device 106 and/or being stored in the storage medium 125 of the wearable device 100) may be combined with any suitable information stored in the storage medium 126 of the alert device 110A (which acts as a server) in order for suitable information to be output on a display (such as display 114 or 118) of an alert device. In one embodiment, the alert information received from the wearable device 100 may comprise an identifier (e.g. a MAC address) of the wearable device 100. The information output for display on each of one or more alert devices is then retrieved from the storage medium 126 of the alert device 110A by the controller 103 and used to generate the information output for display. In the case that the alert information is forwarded by the alert device 110A to one or more other alert devices (such as alert device 1101B), the information retrieved from the storage medium 126 may be included in the forwarded alert information. The relevant information associated with a particular wearable device 100 (e.g. associated with that wearable device's MAC address) may be stored in the storage medium 126 in advance (e.g. during an initial registration process of the wearable device 100) and retrieved from the storage medium 126 using a lookup table stored in the storage medium 126 (the lookup table relating the MAC address of each wearable device 100 with one or more pieces of information associated with that wearable device 100).

One piece of information which may be included in the alert information (and is included in the case of FIG. 2A) is the location of the patient at the time that the predetermined physiological event (in this example, a cardiac arrest). is detected. Such information is very useful, particularly in the case that the physiological event is sufficiently serious for help to be sent to the patient immediately at their current location. The location of the patient is determined by the location circuitry 104 comprised within the wearable device 100. This location circuitry may comprise one or more types of Global Navigation Satellite System (GNSS) circuitry configured to determine the position of the wearable device 100 (and therefore of a patient wearing the wearable device) using satellite signals. An example of such a system is the Global Positioning System (GPS), in which case, the location circuitry 104 is a GPS receiver. The location circuitry 104, instead of or in addition to using a GNSS location method, may be able to determine the location of the wearable device 100 via non-satellite methods, e.g. based on the position of one or more cellular base stations (such technology is known in the art and will therefore not be discussed in detail here).

It will be appreciated that, alternatively, an implanted medical device 106 itself may comprise location circuitry 104 instead of the wearable device 100 comprising the location circuitry 104. In this case, location information generated by the location circuitry 104 is transmitted to the wearable device 100 from the implanted medical device 106 (e.g. as part of information transmitted from the transmitter 108 of the medical device 106 to the receiver 101 of the wearable device via a wireless (e.g. radio) signal). However, by having the location circuitry 104 located in the wearable device 100, the benefits of the alert information comprising real-time location information of the patient are realised without the need for the implanted medical device 106 to comprise the location circuitry 104. In particular, such an arrangement allows existing medical devices 106 such as ICDs which do not include location circuitry 104 to be used with the wearable device 100 whilst still allowing the location of the patient to be included in alert information transmitted by the wearable device 100 in the case that a predetermined physiological event such as a cardiac arrest (in the case of an ICD) occurs.

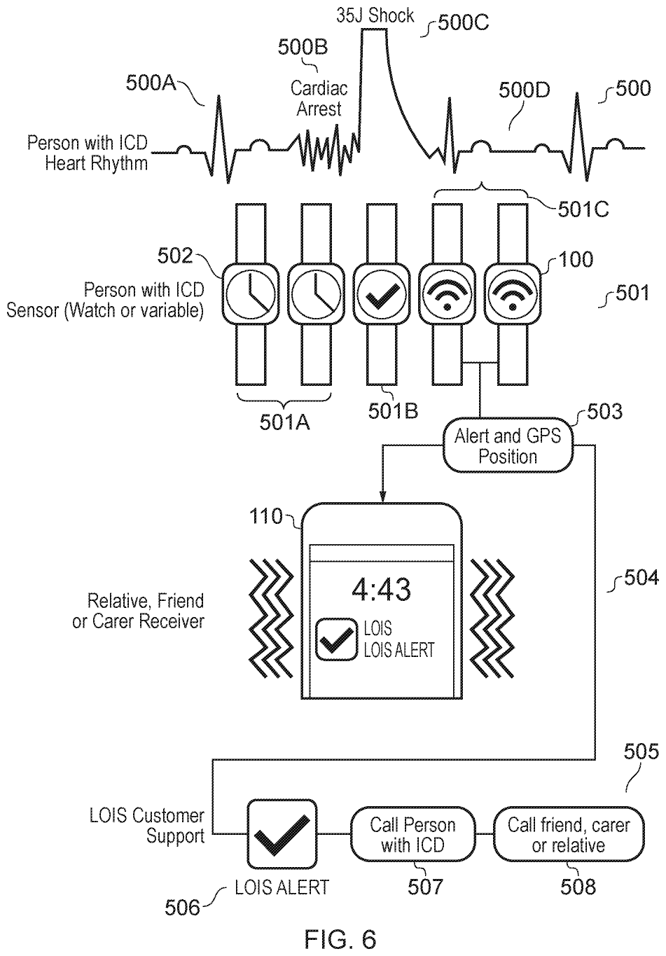

According to an embodiment, FIG. 6 shows a sequence of events which occur when the implanted medical device 106 is an ICD, the wearable device 100 is configured to detect an electrical signal transmitted by the ICD as a result of an electric shock applied to a patient and in which a cardiac arrest occurs. The top portion of FIG. 6 shows an electric cardiogram 500 of the patient. The electric cardiogram is indicative of electrical activity of the patient's heart over time, as detected by the detector 107 of the implanted ICD. Initially, as indicated by 500A, the patient's heart rhythm is normal. As demonstrated by the next portion 501 of FIG. 6, this results in the wearable device 100 (which, in this embodiment, has a display 502 such as a liquid crystal display (LCD) or the like) exhibiting a function in which alert information is not transmitted to an alert device. In this case, as indicated by 501A, the display 502 of the wearable device 100 shows a clock face so as to enable the patient 300 to see the current time, the wearable device 100 therefore performing the function of an ordinary wristwatch. It will be appreciated that the wearable device 100 may further comprise other functions in addition to the core function of detected a signal output by the medical device 106 (such as various calendar, reminder, messaging functions or the like).

At a subsequent time, however, the patient's heart rhythm changes (see point 500B). This is detected by the detector 107 as a dangerous cardiac arrhythmia indicative of a cardiac arrest. In response to his, as indicated by 5000, an electric shock is delivered to the patient by the ICD device. In this case, a 35 joule shock is delivered. The electrode which delivers the shock acts as the transmitter 108 of the ICD and an electrical signal resulting from the application of the electric shock is detected by the wearable device 100 (in particular, by the receiver 101 of the wearable device 100). The wearable device 100 therefore indicates on its display 502 that the electrical signal indicative of the applied shock has been detected. This is indicated at step 501B (in which the information displayed on the display 502 of the wearable device 100 is changed to indicate the detection of the signal transmitter by the ICD). Looking back to the top portion 500 of FIG. 6, it can be seen that the shock applied to the patient has corrected the dangerous arrhythmia and that the patient's heart rhythm has therefore returned to normal. However, because of the application of the shock, the recommendation is that the patient is checked over by a doctor or healthcare professional. Thus, in response to the detection by the receiver 101 of the electric signal, at step 501C, alert information is transmitted by the transmitter 102 of the wearable device 100. In this case, the alert information 503 comprises information indicative of the cardiac arrest occurring together with GPS position information determined by the location circuitry 104 of the wearable device 100. The alert information is received by an alert device 110 (in the form of a smartphone) of a person previously chosen by the patient to receive alerts in the event of a cardiac arrest occurring. For example, this person may be a relative, friend or carer of the patient. This is shown in portion 504 of FIG. 6. In addition to the alert device 110 receiving the alert information, alert information is also transmitted to a customer support service (in particular, to an alert device at a premises of the support service) which alerts an employee of the support service (e.g. a healthcare professional) of the cardiac arrest. This alert occurs at step 506. The employee may then call the patient at step 507 and/or call the relative, friend or carer of the patient at step 508 to find out more information about the incident and to provide advice on what next steps need to be taken. It is noted that, in FIG. 6, the term “LOIS” is an acronym for “Loved Ones Information Service” (highlighting the fact that embodiments of the present technique allow a loved one (such as a partner or family member) of a patient 300 to be notified quickly of medical emergencies detectable by a medical device implanted in the patient 300.

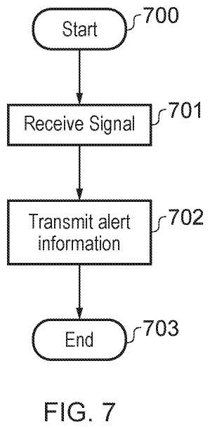

FIG. 7 schematically shows a method of operating the wearable device 100. The method starts at step 700. At step 701, the receiver 101 is controlled to receive a signal from the medical device 106 implanted within the body of a wearer of the wearable device, the signal being transmitted by the implanted medical device in response to detection by the implanted medical device of a predetermined physiological event. At step 702, the transmitter 102 is controlled to transmit, in response to the receiver 101 receiving the signal from the implanted medical device 106, alert information to one or more alert devices for alerting a person other than the wearer of the wearable device of the occurrence of the predetermined physiological event. The method then ends at step 703.

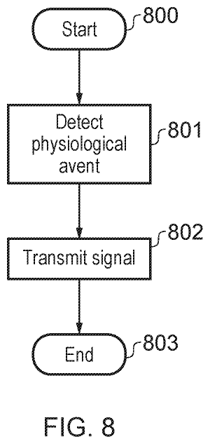

FIG. 8 schematically shows a method of operating the medical device 106, the medical device being implantable in a body of a user. The method starts at step 800. At step 801, the detector 107 is controlled to detect, when the medical device is implanted in a body of a user, a predetermined physiological event. At step 802, the transmitter 108 is controlled to transmit, in response to the detector 107 detecting the predetermined physiological event, a wireless signal to the wearable device 100 worn by the user. The wearable device 100 is configured to transmit, in response to receiving the wireless signal from the implanted medical device, alert information to one or more alert devices for alerting a person other than the wearer of the wearable device of the occurrence of the predetermined physiological event. The process then ends at step 803.

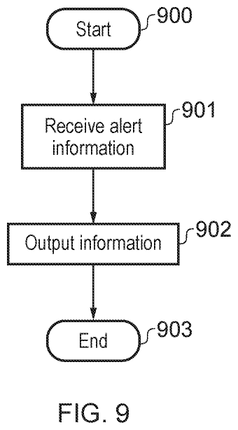

FIG. 9 schematically shows a method of operating an alert device (such as alert device 110A). The method starts at step 900. At step 901, a receiver (such as receiver 111) is controlled to receive, from the wearable device 100 worn by a user, alert information, the alert information being transmitted by the wearable device worn by the user in response to the wearable device receiving a signal transmitted by the medical device 106 implanted within the body of the user in response to the implanted medical device detecting a predetermined physiological event. At step 902, information output circuitry (such as information output circuitry 112) is controlled, in response to the receiver 111 receiving the alert information, to output information indicative of the occurrence of the predetermined physiological event to a person other than the user wearing the wearable device.

In an embodiment, instead of or in addition to the receiver 101, the wearable device 100 comprises an electric field detector configured to detect an electric field generated by the implanted medical device 106 when the implanted medical device 106 performs electrical therapy. For example, the electric field detector may detect the electric field produced by an electric shock delivered by an ICD or may detect the electric field produced by electrical impulses delivered by a pacemaker. The detected electric field is then analysed by the controller 103 and appropriate alert information is transmitted by the transmitter 102 to an alert device 110A or 110B. The alert information comprises information indicative of the therapy delivered and any additional information which may help a healthcare professional or person (such as a friend or relative) previously chosen by the wearer of the wearable device 100 to take appropriate action. For an ICD, the alert information is the same as previously described, for example. For a pacemaker, the alert information comprises a record indicating each time that the pacemaker delivers electrical impulses, for example. This allows suitable analysis of the pacemaker performance over time (e.g. when the patient has a medical check up, the number of occasions on and how often the pacemaker has delivered electrical impulses since the patient's previous medical check up (e.g. 6 months ago) can be analysed to determine whether the pacemaker battery needs to be replaced, whether any pacemaker settings need to be adjusted, whether any additional treatments are suitable for the patient or the like). It will be appreciated that the alert information comprises any suitable information which can be ascertained from the electric field detected by the electric field detector.

FIG. 10 shows a wearable device 1001 according to such an embodiment. The wearable device 1001 is the same as the wearable device 100 except that the receiver 101 is replaced with an electric field detector 1000. Alternatively, the electric field detector 1000 may be present in addition to the receiver 101. The electric field detector 1000 is controlled by the controller 103 (e.g. the controller 103 comprises processing circuitry configured to process instructions for controlling the electric field detector 1000). The electric field detector 1000 is implemented as appropriate circuitry.

Detection of the electric field of an electrical impulse delivered by an implanted medical device 106 (e.g. an ICD shock or electrical impulses from a pacemaker) rather than direct detection of the electrical impulse itself (e.g. by electrodes in direct contact with the patient's skin) allows the occurrence of electrical therapy to be detected when the patient is not wearing the wearable device 1001 (that is, the wearable device 1001 is not in physical contact with the patient) but is merely in sufficient proximity to the wearable device for an electric field generated by the implanted medical device 106 to be detected (e.g. if the patient takes off the wearable device 1001 and puts in on a bedside table next to them as they sleep, therapy such as an ICD shock can still be detected). Furthermore, even if the patient is wearing the wearable device 1001, detection of the electric field is more reliable than direct detection of the electrical impulse (e.g. electrode performance may be adversely affected if the wearable device becomes loose, if clothing gets between the electrode and the patient's skin or in the presence of moisture or dirt, whereas performance of an electric field detector will not be significantly affected in these situations). The detection of electrical therapy and the transmission of alert information thus occurs more reliably via electric field detection. The wearable device 1001 need not even be wearable when electrical therapy is detected using electric field detection. That is, wearable device 1001 may be, more generally, a wearable or non-wearable device having the components of FIG. 10. A non-wearable device may be placed at a location close to which the patient is expected to spend extended periods of time (e.g. next to the patient's bed), for example.

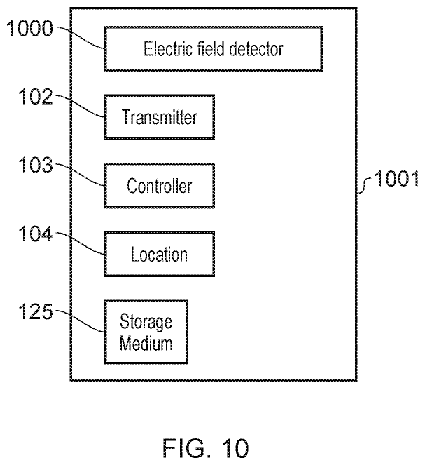

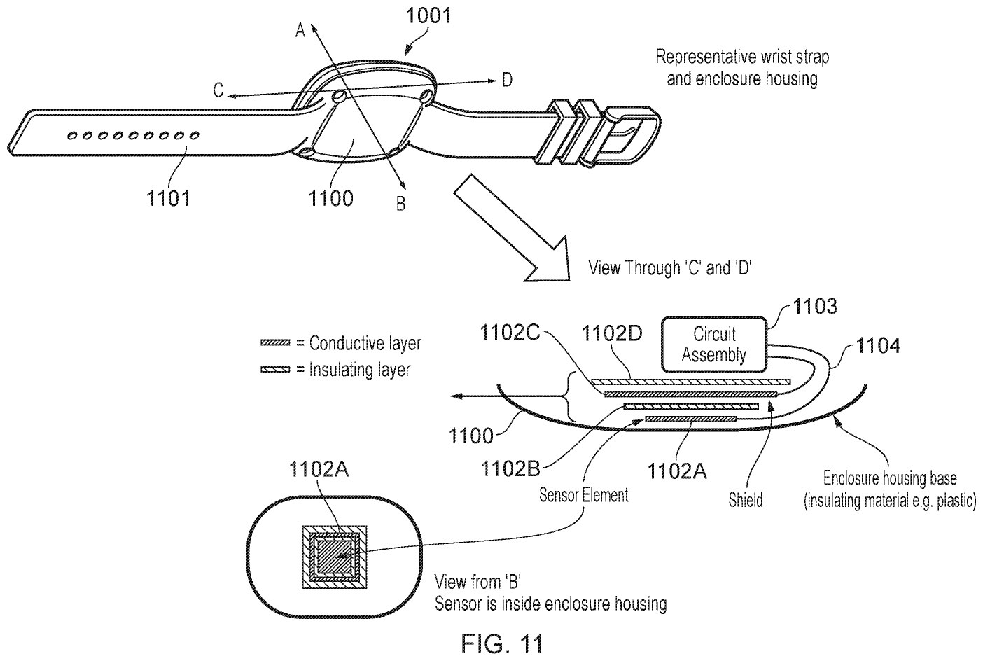

FIG. 11 shows a wearable device 1001 according to an embodiment. The wearable device comprises a housing 1100 (comprising the components of FIG. 10) and a wrist strap 1101 for releasably securing the wearable device to a wrist of the patient. The housing 1100 comprises an electrically insulating material (e.g. plastic) so that electric charge cannot flow between the patient and the electrical components within the housing 1100. The electric field detector 1000 comprises an electrically conductive plate 1102A in electrical communication (via connecting wire 1104) with a circuit assembly 1103 comprising the controller 103. The transmitter 102, location circuitry 104 and storage medium 125 may also be comprised within the circuitry assembly 1103. The plate 1102A is electrically shielded from the circuit assembly 1103 by shield 1102C (which may also take the form of an electrically conductive plate) and is electrically insulated from the shield by an insulating later 110B (made of plastic or the like). The shield 1102C is electrically insulated from the circuitry assembly 1103 by a further insulating later 1102D (made of plastic or the like). The plate 1102A of the electric field detector 1000 may be referred to as a sensor element.

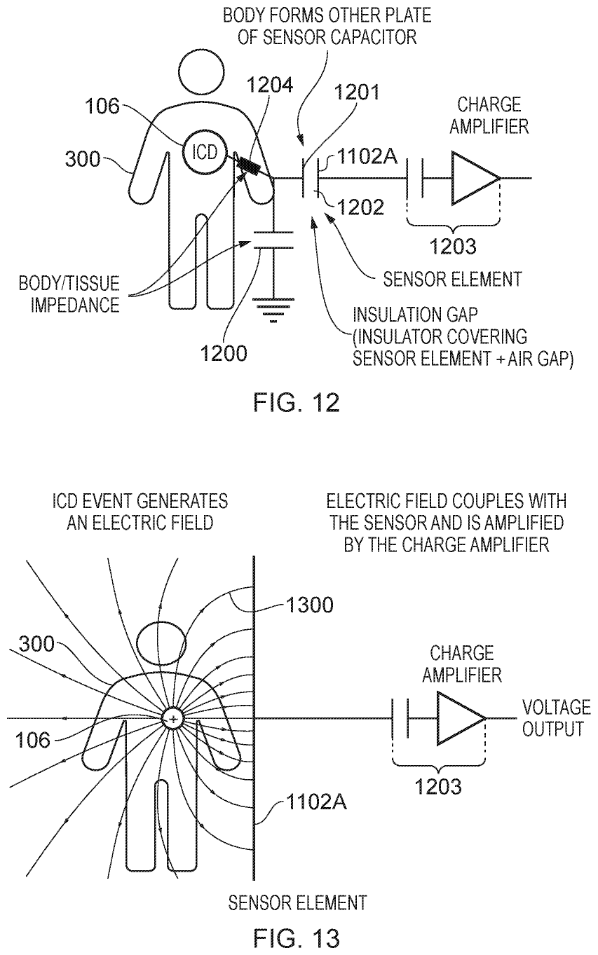

FIG. 12 schematically shows the electrical system formed by the patient 300 and wearable device 1001 when the wearable device is worn by the patient (or, if not worn, in sufficient proximity to the patient so as to detect the electric field provided by a medical device 106 implanted in the patient). The patient's body is electrically grounded and has a natural impedance and capacitance represented by resistor 1204 and capacitor 1200, respectively. The patient's body and sensor element 1102A together form a capacitor in which the patient's body acts as one electrically conductive element 1201 of the capacitor and the sensor element 1102A forms another electrically conductive element of the capacitor. The conductive elements 1201 and 1102A are electrically insulated from each other (thus forming the capacitor) by the electrically insulating material of the housing 1100 and any air gap between the wearable device 1001 and patient 300 and/or between the sensor element 1102A and housing 1100. The sensor element 1102A is electrically connected to a charge amplifier 1203 (comprised as part of the circuit assembly 1103). The charge amplifier outputs a voltage proportional to the integrated value of an input current provided by the sensor element 1102A in response to an electric field which couples to the sensor element 1102A. The voltage generated by the charge amplifier is output to the controller 103. In this way, an electric field generated by therapy delivered by the implanted medical device 106 (in this case, an ICD) is detectable by the controller 103. This is schematically show in FIG. 13, in which the implanted medical device 106 produces an electric field 1300 which couples to the sensor element 1102A. The charge amplifier 1203 then responds to the resulting current by outputting a voltage to the controller 103 which is proportional to an integrated value of the resulting current. The sensor element 1202A (electrically insulated from the patient) and charge amplifier 1203 together form the electric field detector 1000 in this embodiment.

In order to distinguish a detected electric field generated by an implanted medical device from a background electric field in the patient's environment, one or more characteristics of the voltage output by the charge amplifier 1203 are analysed by the controller 103. The one or more analysed electric field characteristics are compared with one or more corresponding known characteristics of the type of medical device implanted in the patient (data indicative of which is stored in the storage medium 125, for example). If there is a match between the one or more analysed and known characteristics, then it is determined that therapy has been delivered by the medical device and suitable alert information is transmitted. On the other hand, if there is not a match between the one or more analysed and known characteristics, then it is determined that therapy has not been delivered by the medical device and no alert information is transmitted. This helps reduce the risk that false alert information is transmitted by the wearable device 1001 (e.g. it helps prevent a detected electric field from the patient's environment being mistakenly identified as the electric field associated with ICD shock). The transmission of false alert information is undesirable since it may result in resources (e.g. an ambulance) being sent to the patient and loved ones being alerted when this is not necessary.

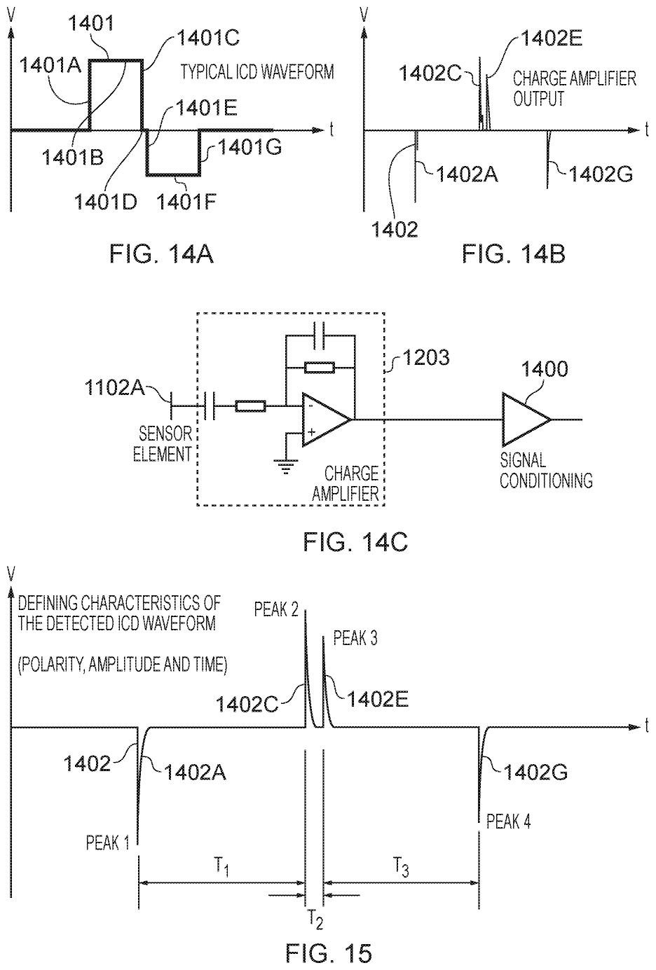

FIGS. 14A to 14C show an example of the voltage signal output by the charge amplifier 1203 in response to delivery of a shock by an ICD. FIG. 14A shows a graph of the temporal voltage output profile 1401 of the ICD when a shock is delivered. The voltage output profile starts at 0V, followed by a step up 1401A, followed a period 1401B of constant voltage delivery, followed by a step down 1401C back to zero, followed by a brief intervening period 1401D of 0V, followed by a further step down 1401E, followed by a period 1401F of constant voltage delivery and followed by a step up 1401G back to 0V. FIG. 14B shows the corresponding temporal voltage output profile 1402 of the charge amplifier 1203. Each step up or step down of the ICD output voltage is indicated by a corresponding peak (with a negative polarity for an ICD voltage step up and negative polarity for an ICD voltage step down) in the charge amplifier output voltage. In particular, a negative peak 1202A corresponds to the step up 1401A, a positive peak 1202C corresponds to the step down 1401C, a positive peak 1402E corresponds to the step down 1401E and a negative peak 1402G corresponds to the step up 1401G. The amplitude of each charge amplifier output voltage peak corresponds to the amplitude of the ICD output voltage step up or step down corresponding to that peak (so that the larger the magnitude of the step up or step down, the large the peak amplitude). FIG. 14C shows, in more detail, the components of the charge amplifier 1203, according to an embodiment (the charge amplifier 1203 has these components even though they are not shown FIGS. 12 and 13). FIG. 14C also shows a signal conditioning element 1400. This forms part of the controller 103 and comprises circuitry for conditioning the output voltage signal of the charge amplifier 1203 so as to allow it to be analysed by the controller 103. The signal conditioning comprises analog-to-digital conversion and, if necessary, filtering and/or further amplification, for example.

Optionally, the signal conditioning comprises polarity inversion. Intuitively, the rising edge 1401A of the ICD signal should give rise to a positive peak, but the charge amplifier is inverting by design and hence a negative peak 1402A results. Likewise, the falling edge 1401C should intuitively create a negative peak, but in reality the inversion causes a positive peak 1402C. Similar observations apply to falling edge 1401E and rising edge 1401G and corresponding peaks 1402E and 1402G. The peak polarities of the ICD signature are thus ‘−++−’ when, intuitively, the peak polarities of the ICD signature should be ‘+−−+’. The more intuitive ‘+−−+’ is achieved by the polarity inversion of the signal conditioning.

Optionally, multiple versions of an electric field signature are stored in the storage medium 125, each different version including a different respective sequence of peak polarities. For example, a stored ICD electric field signature may include a first version with peak polarities ‘−++−’ and a second version with peak polarities ‘+−−+’. This allows the controller 103 to recognise electric fields with the same signature except for different polarities, thus increasing the reliability of the electric field detection. For example, depending on the wiring configuration of a particular ICD, it may produce a ‘+−−+’ polarity sequence or ‘−++−’ polarity sequence. If the signature for each of these polarity sequences is stored and a newly detected electric field is compared to both, then an ICD shock will be detected for either ICD wiring configuration.

The temporal position, number and/or polarity of the charge amplifier output voltage peaks are examples of characteristics which can be analysed by the controller 103 in order to determine whether a detected electric field corresponds to therapy delivered by an implanted medical device 106. This is illustrated in FIG. 15, which shows the peaks of the temporal voltage output profile 1402 of the charge amplifier 1203 in more detail. The occurrence of these four peaks (each indicated by a sharp increase in amplitude magnitude relative to the background output voltage, e.g. as indicated by an increase in amplitude magnitude and a rate of change of amplitude magnitude which exceed respective predefined thresholds), the polarity of the peaks (peaks 1402A and 1402G having a negative polarity and peaks 1402C and 1402E having a positive polarity) and the time between peaks (a time T₁ between peaks 1402A and 1402C, a time T₂ between peaks 1402C and 1402E and a time T₃ between peaks 1402E and 1402G) together form a signature which distinguishes the electric field provided by an ICD shock from other electric fields. That is, the controller 103 knows that an ICD shock has occurred when the charge amplifier output voltage has a peak of negative polarity, followed by a peak of positive polarity a time T₁ later, followed by another peak of positive polarity at a time T₂ later, followed by a peak of negative polarity at a time T₃ later. Other electric fields (e.g. in the background or generated by a static electric shock experienced by the patient) are unlikely to have these specific characteristics, thereby allowing the ICD shock to be distinguished from other electrical activity.

More generally, therapeutic electrical pulses delivered by an implanted medical device (e.g. an ICD shock or electrical pulses delivered by a pacemaker) have a predicable voltage profile which, in turn, results in a predicable charge amplifier output voltage signature like that shown in FIG. 15. Information indicative of the charge amplifier output voltage signature associated with a particular type of implanted medical device is stored in the storage medium 125. When the signature of a newly detected charge amplifier output voltage matches the stored signature of an implanted medical device, the controller 103 determines that the implanted medical device concerned has delivered therapy and causes appropriate alert information to be transmitted. On the other hand, when the signature of a newly detected charge amplifier output voltage does not match the stored signature of an implanted medical device, the controller 103 determines that the detected electrical field is unrelated to an implanted medical device and does not cause any alert information to be transmitted. Such electric fields may simply be present in the environment of the patient or may be the result of a static electric shock or the like experienced by the patient. Such electric fields are very unlikely to have the same behaviour as the highly predicable behaviour of electric fields resulting from therapy provided by an implanted medical device such as an ICD or pacemaker. They are thus unlikely to result in a charge amplifier output voltage profile which matches the signature associated with an implanted medical device. The risk of false alert information being transmitted is thus reduced whilst robust detection of genuine electrical therapy is maintained.

It will be appreciated that determining whether or not the signature of a newly output charge amplifier voltage matches that of an implanted medical device which has been previously stored may be carried out by the controller 103 using any suitable signal processing technique (e.g. any suitable peak detection technique). In an embodiment, the relative timing of peaks (e.g. T₁, T₂ and T₃ in FIG. 15) may have a predetermined tolerance to account for any variation in these timings for different occurrences of therapy delivered by the same implanted medical device. For example, if each of T₁, T₂ and T₃ of a newly detected charge amplifier output are within a predetermined respective percentage tolerance (e.g. ±5%, ±10% or ±20%) of T₁, T₂ and T₃ of the stored signature, then a signature match is determined. The tolerances are determined in advance and stored in the storage medium 125 in accordance with the particular characteristics of the implanted medical device to be monitored, for example. In an embodiment, the controller 103 continuously monitors the output of the charge amplifier in order to determine whether or not electrical therapy has been delivered.

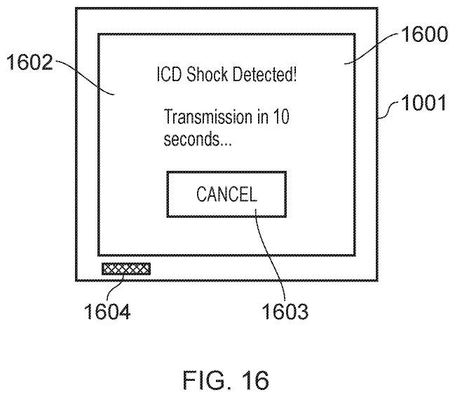

In an embodiment, to further reduce the risk of non-medical device originating electric fields (e.g. static shocks) causing the transmission of false alert information, in response to a charge amplifier output voltage signature match occurring, the patient is temporarily given an option to cancel the transmission of alert information. An example of this is shown in FIG. 16, in which the wearable device 1001 comprises a user interface 1600 in the form of a touch panel display. The touch panel display is controlled by the controller 103. In this example, the charge amplifier output voltage signature has been found to match that of an ICD shock (e.g. like that shown in FIG. 15). In response to this, the touch panel display displays a message 1602 indicating that an ICD shock has been detected and that transmission of alert information will occur after a given cancellation time period. In this case, the cancellation time period is 10 seconds. As time passes, the value “10” is updated to read the amount of time which is left until alert information is transmitted (e.g. 1 second after initially displaying the message 1602, the display shows the value “9”, 2 seconds after initially displaying the message 1602, the display shows the value “8”, and so on, in a count down manner). After 10 seconds has elapsed, the alert information is transmitted unless, during the 10 second period, the user touches the “Cancel” virtual button 1603.

Instead of or in addition to the display of the message 1602, a loudspeaker 1604 of the wearable device 1001 (also controlled by the controller 103) may provide an audible alarm during the 10 second period to alert the user that alert information is to be transmitted. The audible alarm is sounded until the “Cancel” virtual button 1603 is pressed (in which case, no alert information is transmitted) or until the 10 second period has elapsed (at which point, alert information is transmitted).

It will be appreciated that, instead of a touch panel display, the user interface 1600 may comprise other hardware for outputting visual information to the user (e.g. a non-touch panel display or one or more light emitting elements such as one or more light emitting diodes (LEDs) which are controlled to flash at an increasing rate the closer it is to the time at which the 10 second time period elapses) and receiving an input from the user to cancel the transmission of alert information (e.g. a physical button, switch or the like).

Thus, in the case that a false match of the charge amplifier output voltage signature occurs, the user is given the opportunity to cancel the transmission of alert information. This is particularly applicable to detection of an ICD shock. Occurrence of an ICD shock is very obvious to a patient within which the ICD is implanted. It will therefore be obvious to the patient if they see and/or hear the wearable device 1001 warn that alert information is to be transmitted and they have not experienced such a shock (this may occur if the user experiences a static shock which results in a charge amplifier output voltage signature which happens to match the signature of an ICD shock, for example). The cancellation time period during which the user may cancel transmission of the alert information is set so as to give the user sufficient time to issue such a cancellation in the case of a false signature match but not so long as to unduly delay the transmission of alert information (and therefore help being provided to the user) in the case of a genuine ICD shock. For example, the cancellation time period may be 10, 20, 30 or 60 seconds.

In the above-mentioned examples, the charge amplifier output voltage signature is that of an ICD shock. It will be appreciated that other types of implanted medical devices 106 which delivery electrical therapy to a patient by applying one or more pulses of electrical energy to the patient's body in a predicable way (so as to have predicable electric field behaviour and therefore a predictable charge amplifier output voltage signature) may also be used with the present technique. A pacemaker is such an example. A pacemaker delivers electrical impulses with a predicable voltage profile to a patient's heart in order to regulate the heart's electrical conduction system. Each electrical impulse is therefore associated with the same charge amplifier output voltage signature. The electrical impulses delivered by a pacemaker have far less energy than an ICD electric shock. However, as long as the electric field generated by each electric impulse from the pacemaker is detectable by the sensor element 1202A and can be classified by the controller 103 based on the output of the charge amplifier, the electrical therapy delivered by the pacemaker is detectable. The detection of electrical therapy delivered by the pacemaker may be used by the controller 103 to generate information on the pacemaker performance (e.g. how many times and/or how often the pacemaker delivered therapy in a given time period). This information may then be transmitted as alert information (e.g. for storage and/or analysis in a central server). Alternatively or in addition, the information may be stored in the storage medium 125 for later analysis (e.g. for download to a patient or doctor's computer or other device (such as a smart phone) for analysis).

Like the ICD signature, the signature of another implanted medical device 106 (such as a pacemaker) is defined by one or more predetermined features of the charge amplifier voltage output profile (e.g. the number of peaks, the polarity of each peak and/or the time period between peaks) which are characteristic of the electric field generated by that implanted medical device 106 when it performs therapy. Information indicative of the signature of the implanted medical device which is to be detected is stored in the storage medium 125. A newly captured charge amplifier output voltage profile is then compared with the stored signature. If there is a match, then it is determined that the implanted medical device has performed therapy and an appropriate action is taken (e.g. transmission of alert information). On the other hand, if there is no match, then the appropriate action is not taken.

The present technique can also be used to detect other types of electric field by detecting a signature of that electric field and comparing it to a stored electric field signature. In this case, the wearer need not be a patient with an implanted medical device. For example, an electric field of a shock from mains electricity is detected and, in response, the transmitter 102 transmits a signal indicating that such a shock has occurred and a location determined by the location circuitry 104. This allows emergency medical help to be sent to the wearer of the wearable device 1001. The wearer may be a construction or electrical worker, for example (who are at risk of mains electric shocks when working). In another example, an electric field of an electrostatic discharge is detected and, in response, the transmitter 102 transmits a signal indicating that the electrostatic discharge has occurred and information indicative of an identifier of the wearer of the wearable device 1001 (e.g. an employee number stored in the storage medium 125). The wearer may be a worker in a factor making products (e.g. electronics) which are sensitive to electrostatic discharge, for example. This allows products potentially affected by an electrostatic discharge to be identified during production (e.g. if an electrostatic discharge is detected then it is concluded that the product currently being handled by the worker may have been affected). Potentially affected products can then be tested to ensure they function correctly. This improves quality control whilst alleviating the need to test every product coming off the production line. Other example uses are also envisaged, as long as they involve detection of an electric field in the way described.

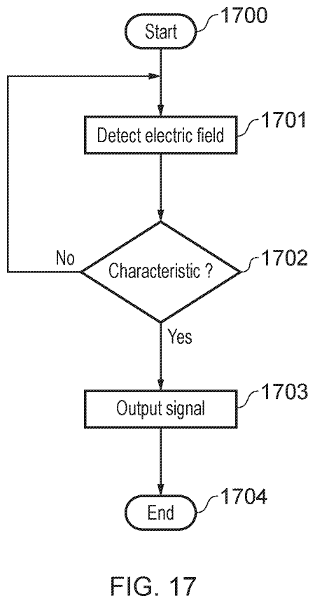

FIG. 17 shows a method carried out by the controller 103 of a device (e.g. wearable device 1001 or a non-wearable device) according to an embodiment. The method starts at step 1700. At step 1701, the electric field detector 1000 is controlled to detect an electric field (e.g. by switching the charge amplifier 1203 into an operational state when the electric field detector 1000 comprises a sensor element 1102A and charge amplifier 1203). At step 1702, it is determined if the detected electric field has a characteristic indicative of a predetermined electric field source (e.g. an electric shock delivered by an ICD, a series of electrical impulses delivered by a pacemaker, a mains electric shock or an electrostatic discharge). If the detected electric field does not have such a characteristic, then the method returns to step 1701. If the detected electric field does have such a characteristic, then, at step 1703, a signal is output indicating that the predetermined electric field source has been detected. The signal is output to the transmitter 102 (in response to which the transmitter transmits alert information) or the storage medium 125 (in response to which the storage medium stores information indicating that the predetermined electric field source was detected), for example. The method ends at step 1704.

Embodiments of the present technique are defined by the following numbered clauses:

1. A wearable device comprising:
receiver circuitry configured to receive a signal from a medical device implanted within the body of a wearer of the wearable device, the signal being transmitted by the implanted medical device in response to detection by the implanted medical device of a predetermined physiological event; and
transmitter circuitry configured to transmit, in response to the receiver circuitry receiving the signal from the implanted medical device, alert information to one or more alert devices for alerting a person other than the wearer of the wearable device of the occurrence of the predetermined physiological event.

2. A wearable device according to clause 1, wherein the signal received from the implanted medical device is a wireless signal.

3. A wearable device according to clause 1, wherein the signal received from the implanted medical device is an electric signal transmitted from the implanted medical device to the wearable device via organic tissue of the wearer of the wearable device.

4. A wearable device according to clause 3, wherein:
the implanted medical device is an Implantable Cardioverter Defibrillator (ICD);
the predetermined physiological event is a cardiac arrhythmia indicative of a potential cardiac arrest; and
the source of the electric signal received by the receiver circuitry is an electric shock generated by the ICD for alleviating the cardiac arrhythmia indicative of a potential cardiac arrest.

5. A wearable device according to any preceding clause, wherein the transmitter circuitry is configured to transmit the alert information to the one or more alert devices via a communications network.

6. A wearable device according to clause 5, wherein the communications network comprises a mobile telecommunications network.

7. A wearable device according to clause 5 or 6, wherein the communications network comprises a local area network (LAN).

8. A wearable device according to any preceding clause, wherein the alert information comprises data associated with the detected predetermined physiological event generated based the signal received from the implanted medical device.

9. A wearable device according to clause 8, wherein the data associated with the detected predetermined physiological event comprised within the alert information comprises information indicative of a location of the wearer of the wearable device.

10. A wearable device according to clause 9, comprising location circuitry configured to determine the location of the wearer.

11. A wearable device according to any preceding clause, wherein one of the one or more alert devices is for alerting a healthcare professional of the occurrence of the predetermined physiological event.

12. A wearable device according to any preceding clause, wherein one of the one or more alert devices is for alerting a person previously chosen by the wearer of the wearable device of the occurrence of the predetermined physiological event.

13. A medical device implantable in a body of a user, the medical device comprising:
detection circuitry configured to detect, when the medical device is implanted in a body of a user, a predetermined physiological event; and
transmitter circuitry configured to transmit, in response to the detection circuitry detecting the predetermined physiological event, a wireless signal to a wearable device worn by the user, the wearable device being configured to transmit, in response to receiving the wireless signal from the implanted medical device, alert information to one or more alert devices for alerting a person other than the wearer of the wearable device of the occurrence of the predetermined physiological event.

14. An alert device comprising:
receiver circuitry configured to receive, from a wearable device worn by a user, alert information, the alert information being transmitted by the wearable device worn by the user in response to the wearable device receiving a signal transmitted by a medical device implanted within the body of the user in response to the implanted medical device detecting a predetermined physiological event; and
information output circuitry configured, in response to the receiver circuitry receiving the alert information, to output information indicative of the occurrence of the predetermined physiological event to a person other than the user wearing the wearable device.

15. An alert device according to clause 14, wherein the person other than the user wearing the wearable device is a healthcare professional.

16. An alert device according to clause 14, wherein the person other than the user wearing the wearable device is a person previously chosen by the wearer of the wearable device.

17. A method of operating a wearable device comprising receiver circuitry and transmitter circuitry, the method comprising:
controlling the receiver circuitry to receive a signal from a medical device implanted within the body of a wearer of the wearable device, the signal being transmitted by the implanted medical device in response to detection by the implanted medical device of a predetermined physiological event; and
controlling the transmitter circuitry to transmit, in response to the receiver circuitry receiving the signal from the implanted medical device, alert information to one or more alert devices for alerting a person other than the wearer of the wearable device of the occurrence of the predetermined physiological event.

18. A method of operating a medical device implantable in a body of a user, the medical device comprising detection circuitry and transmitter circuitry, wherein the method comprises:
controlling the detection circuitry to detect, when the medical device is implanted in a body of a user, a predetermined physiological event; and
controlling the transmitter circuitry to transmit, in response to the detection circuitry detecting the predetermined physiological event, a wireless signal to a wearable device worn by the user, the wearable device being configured to transmit, in response to receiving the wireless signal from the implanted medical device, alert information to one or more alert devices for alerting a person other than the wearer of the wearable device of the occurrence of the predetermined physiological event.

19. A method of operating an alert device, the alert device comprising receiver circuitry and information output circuitry, wherein the method comprises:
controlling the receiver circuitry to receive, from a wearable device worn by a user, alert information, the alert information being transmitted by the wearable device worn by the user in response to the wearable device receiving a signal transmitted by a medical device implanted within the body of the user in response to the implanted medical device detecting a predetermined physiological event; and
controlling the information output circuitry, in response to the receiver circuitry receiving the alert information, to output information indicative of the occurrence of the predetermined physiological event to a person other than the user wearing the wearable device.

20. A program for controlling a computer to perform a method according to any one of clauses 17 to 19.

21. A storage medium storing a program according to clause 20.

22. A monitoring device comprising:
electric field detection circuitry configured to detect an electric field; and processing circuitry configured to:
determine whether the detected electric field has a characteristic indicative of a predetermined electric field source; and
if the detected electric field has the characteristic, output a signal indicating that the predetermined electric field source has been detected.

23. A monitoring device according to clause 22, wherein:
the electric field detection circuitry comprises a conductor electrically connected to a charge amplifier and electrically insulated from a user; and
the processing circuitry is configured to monitor an output of the charge amplifier to determine whether the detected electric field has the characteristic.

24. A monitoring device according to clause 23, wherein the characteristic comprises one or more of a number of peaks of the charge amplifier output, a polarity of one or more peaks of the charge amplifier output and a time between peaks of the charge amplifier output.

25. A monitoring device according to one of clauses 22 to 24, comprising a user interface configured to receive a cancellation input from a user, wherein:
in response to determining that the detected electric field has the characteristic, the processing circuitry is configured to:
determine if a cancellation input is received from the user within a predetermined time period;
if a cancellation input is not received from the user within the predetermined time period, output the signal indicating that the predetermined electric field source has been detected; and
if a cancellation input is received from the user within the predetermined time period, not output the signal indicating that the predetermined electric field source has been detected.

26. A monitoring device according to any one of clauses 22 to 25, wherein the predetermined electric field source is therapy delivered by a medical device implanted within a user.

27. A monitoring device according to clause 26, wherein:
the medical device is an Implantable Cardioverter Defibrillator, ICD; and
the therapy delivered by the ICD is an electric shock for alleviating cardiac arrhythmia indicative of a potential cardiac arrest.

28. A monitoring device according to clause 26, wherein:
the medical device is a pacemaker; and
the therapy delivered by the pacemaker is a series of electrical impulses to regulate the electrical conduction system of the user's heart.

29. A monitoring device according to any one of clauses 22 to 25, wherein the predetermined electric field source is a mains electricity shock.

30. A monitoring device according to any one of clauses 22 to 25, wherein the predetermined electric field source is an electrostatic discharge.

31. A monitoring device according to any one of clauses 22 to 30, comprising:
transmitter circuitry configured to transmit, in response to the output signal indicating that the predetermined electric field source has been detected, alert information to one or more alert devices for alerting a party other than a user of the monitoring device that the predetermined electric field source has been detected.

32. A monitoring device according to clause 31, wherein the transmitter circuitry is configured to transmit the alert information to the one or more alert devices via a communications network.

33. A monitoring device according to clause 32, wherein the communications network comprises a mobile telecommunications network.

34. A monitoring device according to clause 32 or 33, wherein the communications network comprises a local area network (LAN).

35. A monitoring device according to any one of clauses 31 to 34, wherein the alert information comprises data associated with the detected predetermined electric field source.

36. A monitoring device according to clause 35, wherein the data associated with the detected predetermined electric field source comprises information indicative of a location of the user of the monitoring device.

37. A monitoring device according to clause 36, comprising location circuitry configured to determine the location of the user of the monitoring device.

38. A monitoring device according to any one of clauses 31 to 37, wherein one of the one or more alert devices is for alerting a healthcare professional that the predetermined electric field source has been detected.

39. A monitoring device according to any one of clauses 31 to 38, wherein one of the one or more alert devices is for alerting a person previously chosen by the user of the monitoring device that the predetermined electric field source has been detected.

40. A monitoring device according to any one of clauses 22 to 39, wherein the patient monitoring device is wearable by a user of the monitoring device.

41. A monitoring method comprising:
detecting an electric field; and
determining whether the detected electric field has a characteristic indicative of predetermined electric field source; and
if the detected electric field has the characteristic, outputting a signal indicating that the predetermined electric field source has been detected.

42. A program for controlling a computer to perform a method according to clause 41.

43. A storage medium storing a program according to clause 42.

Numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

1. A monitoring device comprising:
electric field detection circuitry configured to detect an electric field; and
processing circuitry configured to:
determine whether the detected electric field has a characteristic indicative of a predetermined electric field source; and
if the detected electric field has the characteristic, output a signal indicating that the predetermined electric field source has been detected;
wherein:
the electric field detection circuitry comprises a conductor electrically connected to a charge amplifier and electrically insulated from a user;
the processing circuitry is configured to monitor an output of the charge amplifier to determine whether the detected electric field has the characteristic; and
the characteristic comprises one or more of a number of peaks of the charge amplifier output, a polarity of one or more peaks of the charge amplifier output and a time between peaks of the charge amplifier output.

2. A monitoring device according to claim 1, comprising a user interface configured to receive a cancellation input from a user, wherein:
in response to determining that the detected electric field has the characteristic, the processing circuitry is configured to:
determine if a cancellation input is received from the user within a predetermined time period;
if a cancellation input is not received from the user within the predetermined time period, output the signal indicating that the predetermined electric field source has been detected; and
if a cancellation input is received from the user within the predetermined time period, not output the signal indicating that the predetermined electric field source has been detected.

3. A monitoring device according to claim 1, wherein the predetermined electric field source is therapy delivered by a medical device implanted within a user.

4. A monitoring device according to claim 3, wherein:
the medical device is an Implantable Cardioverter Defibrillator, ICD; and
the therapy delivered by the ICD is an electric shock for alleviating cardiac arrhythmia indicative of a potential cardiac arrest.

5. A monitoring device according to claim 3, wherein:
the medical device is a pacemaker; and
the therapy delivered by the pacemaker is a series of electrical impulses to regulate the electrical conduction system of the user's heart.

6. A monitoring device according to claim 1, wherein the predetermined electric field source is a mains electricity shock.

7. A monitoring device according to claim 1, wherein the predetermined electric field source is an electrostatic discharge.

8. A monitoring device according to claim 1, comprising:
transmitter circuitry configured to transmit, in response to the output signal indicating that the predetermined electric field source has been detected, alert information to one or more alert devices for alerting a party other than a user of the monitoring device that the predetermined electric field source has been detected.

9. A monitoring device according to claim 8, wherein the transmitter circuitry is configured to transmit the alert information to the one or more alert devices via a communications network.

10. A monitoring device according to claim 9, wherein the communications network comprises a mobile telecommunications network.

11. A monitoring device according to claim 9, wherein the communications network comprises a local area network (LAN).

12. A monitoring device according to claim 8, wherein the alert information comprises data associated with the detected predetermined electric field source.

13. A monitoring device according to claim 12, wherein the data associated with the detected predetermined electric field source comprises information indicative of a location of the user of the monitoring device.

14. A monitoring device according to claim 13, comprising location circuitry configured to determine the location of the user of the monitoring device.

15. A monitoring device according to claim 8, wherein one of the one or more alert devices is for alerting a healthcare professional that the predetermined electric field source has been detected.

16. A monitoring device according to claim 8, wherein one of the one or more alert devices is for alerting a person previously chosen by the user of the monitoring device that the predetermined electric field source has been detected.

17. A monitoring device according to any claim 1, wherein the patient monitoring device is wearable by a user of the monitoring device.

18. A monitoring method comprising:
detecting an electric field; and
determining whether the detected electric field has a characteristic indicative of predetermined electric field source; and
if the detected electric field has the characteristic, outputting a signal indicating that the predetermined electric field source has been detected;
wherein:
the electric field is detected using a conductor electrically connected to a charge amplifier and electrically insulated from a user;
determining whether the detected electric field has the characteristic comprises monitoring an output of the charge amplifier; and
the characteristic comprises one or more of a number of peaks of the charge amplifier output, a polarity of one or more peaks of the charge amplifier output and a time between peaks of the charge amplifier output.

19. A program for controlling a computer to perform a method according to claim 18.

20. A storage medium storing a program according to claim 19.
